(19) United States
(12) Patent Application Publication
(10) Pub. No.: US 2012/0263770 A1
(43) Pub. Date: Oct. 18, 2012

(54) ANTI-ATOPIC DERMATITIS TEXTILES

(76) Inventor: Bong Ja LEE, Wonju-si (KR)

(21) Appl. No.: 13/102,467

(22) Filed: May 6, 2011

(30) Foreign Application Priority Data
Apr. 15, 2011 (KR) 10-2011-0035164

(51) Int. Cl. A61K 9/70; A61P 17/00; A61K 35/56

(52) U.S. Cl. 424/402; 424/520

(57) ABSTRACT

The present invention relates to anti-atopic textiles, including a textile body and skin care layers formed by dyeing the textile body with a dyebath, obtained by filtering a mixture of earthworm powder and water through Korean paper. When clothes are made of the anti-atopic textiles and brought in contact with the skin, atopic dermatitis can be significantly alleviated because a moisturizing action and an anti-inflammation action are accelerated by a body composition and fluid components of earthworms included in the skin care layers and paper mulberry extract mixed from Korean paper. The anti-atopic textiles according to the present invention includes a textile body and skin care layers formed by dyeing the textile body with a dyebath which is obtained by mixing earthworm powder, obtained by pulverizing earthworms and drying the pulverized earthworms, and water and then filtering the mixture through Korean paper.

### CROSS-REFERENCE TO RELATED APPLICATION

Priority is claimed to Korean patent application number 10-2011-0035164 filed on Apr. 15, 2011, the entire disclosure of which is incorporated by reference herein.

### BACKGROUND OF THE INVENTION

1. Field of the Invention

The present invention relates to anti-atopic textiles and, more particularly, to anti-atopic textiles, including a textile body and skin care layers formed by dyeing the textile body with a dyebath, obtained by filtering a mixture of earthworm powder and water through Korean paper, wherein when clothes are made of the anti-atopic textiles and brought in contact with the skin, atopic dermatitis can be significantly alleviated because a moisturizing action and an anti-inflammation action are accelerated by a body composition and fluid components of earthworms included in the skin care layers and paper mulberry extract mixed from Korean paper.

2. Background of the Related Art

In general, atopic dermatitis is a chronic and recurrent inflammatory skin disease which is chiefly started in the infancy or the childhood and is accompanied by pruritus (i.e., itching) and skin xeransis and characteristic eczema.

The etiologies of atopic dermatitis have not yet been precisely known and thus cannot be described using any one cause because there are various clinical symptoms, such as skin xeransis and eczema. However, major causes, such as environmental factors, a genetic predisposition, an immunological reaction, and the abnormality of a skin protection film, are considered as the etiologies of atopic dermatitis. The environmental factors may include environmental pollution, such as smoke due to industrialization, an increase in the use of food additives, an increase in the use of a carpet, bed, and sofa due to westernized housing, and an increase of substance to cause allergy, such as dermatophagoides pteronyssinus due to a rise of indoor temperature. A cause to expose cause substance may become one of the etiologies of atopic dermatitis because pet animals are raised in the room.

One of methods of alleviating atopic dermatitis may include skin moisturizing and inflammatories. The skin moisturizing is chiefly performed using medicines for the skin or a skin moisturizing agent. However, attempts to alleviate atopic dermatitis using textiles directly coming into contact with the skin have not been made a lot.

Clothes using fabrics dyed with vegetable natural dyes or clothes using fabrics dyed with yellow earth have recently been in the spotlight but are problematic in that they do not significantly alleviate atopic dermatitis through a direct skin moisturizing or anti-inflammation action.

### SUMMARY OF THE INVENTION

Accordingly, the present invention has been made in view of the above problems occurring in the prior art, and it is an object of the present invention to provide anti-atopic textiles, including a textile body and skin care layers formed by dyeing the textile body with a dyebath, obtained by filtering a mixture of earthworm powder and water through Korean paper, wherein when clothes are made of the anti-atopic textiles and brought in contact with the skin, atopic dermatitis can be significantly alleviated because a moisturizing action and an anti-inflammation action are accelerated by a body composition and fluid components of earthworms included in the skin care layers and paper mulberry extract mixed from Korean paper.

The above object of the present invention is achieved by anti-atopic textiles, including a textile body and skin care layers formed by dyeing the textile body with a dyebath which is obtained by mixing earthworm powder, obtained by pulverizing earthworms and drying the pulverized earthworms, and water and then filtering the mixture through Korean paper.

### BRIEF DESCRIPTION OF THE DRAWINGS

Further objects and advantages of the invention can be more fully understood from the following detailed description taken in conjunction with the accompanying drawings in which:

FIG. 1 is a cross-sectional view of anti-atopic textiles according to the present invention; and

FIG. 2 is a flowchart illustrating a method of fabricating the anti-atopic textiles according to the present invention.

### DESCRIPTION OF REFERENCE NUMERALS OF PRINCIPAL ELEMENTS IN THE DRAWINGS

1: anti-atopic textiles according to the present invention
10: textile body
20: skin care layers

### DETAILED DESCRIPTION OF THE PREFERRED EMBODIMENTS

Some embodiments of the present invention will now be described in detail with reference to the accompanying drawings, but the embodiments are described in detail to the extent that those skilled in the art to which the present invention pertains can readily implement the embodiments. Accordingly, the technical spirit and category of the present invention are not limited to the embodiments.

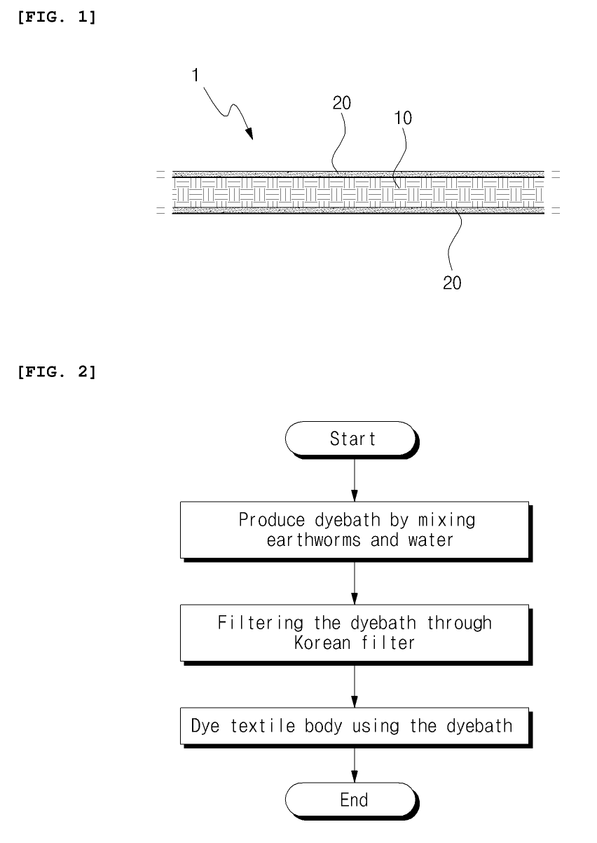

FIG. 1 is a cross-sectional view of anti-atopic textiles according to the present invention, and FIG. 2 is a flowchart illustrating a method of fabricating the anti-atopic textiles according to the present invention.

Referring to FIGS. 1 and 2, the anti-atopic textiles 1 according to the present invention includes a textile body 10 and skin care layers 20 formed by dyeing the textile body 10 with a dyebath, obtained by filtering a mixture of earthworm powder and water through Korean paper. When clothes are made of the anti-atopic textiles 1 and brought in contact with the skin, atopic dermatitis can be significantly alleviated because a moisturizing action and an anti-inflammation action are accelerated by the body composition and the fluid components of earthworms, included in the skin care layers, and paper mulberry extract mixed from Korean paper. As shown in FIG. 1, the anti-atopic textiles 1 includes the textile body 10 and the skin care layers 20, formed by dyeing the textile body 10 with the dyebath obtained by pulverizing earthworms, mixing dried earthworm powder and water, and filtering the mixture through Korean paper.

The textile body 10 is weaved by crossing warps and wefts of various materials. The textile body 10 includes all kinds of fabrics which may be fabricated into clothes, including underwear, upper and lower wear, and pants.

The skin care layers 20 are formed on the textile body 10 through dyeing. The skin care layers 20 function to accelerate the moisturizing action and the anti-inflammation action, when the anti-atopic textiles 1 according to the present invention are fabricated into clothes and the clothes come in contact with the skin. The skin care layers 20 are formed by dyeing the textile body 10 with the dyebath which is obtained by pulverizing earthworms, mixing dried earthworm powder and water, and then filtering the mixture through Korean paper.

It is preferred that the earthworm powder be obtained by collecting earthworms, putting the collected earthworms in an aqueous solution containing a very small amount (0.3 weight % or less) of a chemical compound, consisting of one or more kinds selected from the group consisting of organic acids, inorganic acids, organic acid sodium salt, inorganic acid sodium salt, organic acid potassium, and inorganic acid potassium, or in water having a temperature of 4 to 28 degrees Celcius for a long time (10 to 30 hours) so that all vermicompost within the alimentary canal is excreted, pulverizing the resulting earthworms, and then drying (freeze-drying or vacuum drying) the pulverized earthworms.

It is preferred that the dyebath in which the earthworm powder and water are mixed be obtained by mixing the water of 500 to 1500 weight % and the earthworm powder of 80 to 100 weight % in a temperature range of 40 to 60 degrees Celcius and, at the same time, adding mordant of 8 to 10 weight %, such as salt or alum, to the mixture, stirring the mixture, and then filtering the mixture through Korean paper.

As is known in the art, Korean paper or “hanji” is made from the inner bark of paper mulberry trees that are native to Korean that grow well on its rocky mountainsides. In the process of filtering the dyebath using the earthworm powder as dyes through Korean paper, pharmacological components, such as kazinol, are extracted from paper mulberry fibroid material included in the Korean paper and then included in the dyebath. Accordingly, the pharmacological components, such as kazinol, can be adhered to the textile body 10 while the textile body 10 is dyed, and thus can be provided to a user's skin. In particular, kazinol has been known to be effective in atopic dermatitis caused by dermatophagoides pteronyssinus, as well as skin whitening.

The mordant is cationized while being melt in water, and thus the mordant functions to adhere earthworm powder particles, having weak anion properties, to the textile body 10. Consequently, the mordant functions to improve dyeing force.

The skin care layers 20 is formed by a common dyeing method of several times performing a process of putting the textile body in the dyebath obtained by the above method, soaking and stirring the care layers 20 in the dyebath for 5 to 30 minutes so that the earthworm powder particles are firmly adhered to the textile body 10 uniformly, removing moisture from the anti-atopic textiles 1, and drying the anti-atopic textiles 1 in the shade.

### Embodiment 1

A process of soaking the textile body 10 formed of cotton textiles in the dyebath, formed by mixing the earthworm powder of 1 kg and 50 degrees Celcius water of 10 kg and, at the same time, adding alum of 0.1 kg to the mixture, stirring the mixture, and then filtering the mixture through Korean paper, for 20 minutes, fingering the resulting textile body 10 by the hand, removing moisture from the textile body 10, and then drying the resulting textile body 10 in the shade is repeatedly performed four times. Next, the anti-atopic textiles 1 dyed according to the present invention are fabricated into nightclothes. 10 men and 10 women having atopic dermatitis symptoms wore the nightclothes for 20 days when sleeping.

As a result of a survey after wearing the nightclothes, 6 of the 10 men and 5 of the 10 women told that the frequency of itching due to atopic dermatitis had been significantly reduced. 2 of the 10 men and 3 of the 10 women told that the frequency of itching due to atopic dermatitis had been slightly reduced. 1 of the 10 men told that the frequency of itching due to atopic dermatitis had no change. 1 of the 10 men and 1 of the 10 women told that the frequency of itching due to atopic dermatitis had become worse. From the above survey, it was found that the anti-atopic textiles 1 according to the present invention had effects 80% or higher.

In the anti-atopic textiles 1 according to the present invention, the skin care layers 20 are formed by dyeing the textile body 10 by using the earthworm powder as dyes. When clothes are made of the anti-atopic textiles 1 and brought in contact with the skin, symptoms due to atopic dermatitis can be significantly alleviated because a moisturizing action and an anti-inflammation action are accelerated by the body composition and the fluid components of earthworms, included in the skin care layers 20, and paper mulberry extract mixed from Korean paper.

Furthermore, in the anti-atopic textiles 1 according to the present invention, in the process of filtering the dyebath, using the earthworm powder as dyes, through Korean paper, pharmacological components, such as kazinol, are extracted from paper mulberry fibroid material included in the Korean paper, included in the dyebath, and deposited in the textile body 10. When clothes are made of the anti-atopic textiles 1 according to the present invention and worn by a person, atopic dermatitis can be alleviated and the skin can also be whitened.

As described above, according to the anti-atopic textiles in accordance with the present invention, the skin care layers are formed by dyeing the textile body 10 by using the earthworm powder as dyes. When clothes are made of the anti-atopic textiles and brought in contact with the skin, there is an excellent effect in that atopic dermatitis can be significantly alleviated because a moisturizing action and an anti-inflammation action are accelerated by the body composition and the fluid components of earthworms, included in the skin care layers, and paper mulberry extract mixed from Korean paper.

Furthermore, according to the anti-atopic textiles in accordance with the present invention, in the process of filtering the dyebath, using the earthworm powder as dyes, through Korean paper, pharmacological components, such as kazinol, are extracted from paper mulberry fibroid material included in the Korean paper, and then deposited in the textile body. When clothes are made of the anti-atopic textiles according to the present invention and worn by a person, there is an excellent effect in that atopic dermatitis can be alleviated and the skin can also be whitened.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. Anti-atopic textiles, comprising:
a textile body; and
skin care layers formed by dyeing the textile body with a dyebath which is obtained by mixing earthworm powder, obtained by pulverizing earthworms and drying the pulverized earthworms, and water and then filtering the mixture through Korean paper.

2. A method of making an anti-atopic textile, said method comprising:
pulverizing earthworms;
drying the pulverized earthworms to form an earthworm powder;
mixing the earthworm powder with a liquid to form a mixture;
passing the mixture through a paper filter containing paper mulberry fibroid material to form a dyebath; and
dyeing a textile material with the dyebath.
